SEQUENCE LISTING (1 sequence)

SEQ ID NO: 1 - protein, 40 aa, Artificial Sequence
  Synthetic Peptide
MKVLIVLLAIFAALPLALAQPVINTTVGSAAEGSLDKREA

(19) United States
(12) Patent Application Publication
(10) Pub. No.: US 2011/0059514 A1
(43) Pub. Date: Mar. 10, 2011

(54) Yeast Biocatalysts for Degradation of Biowarfare Agents

(76) Inventors: David Alexander Schofield, Hollywood, SC (US); Augustine Anthony Dinovo, James Island, SC (US)

(21) Appl. No.: 12/522,233

(22) PCT Filed: Jan. 7, 2008
(86) PCT No.: PCT/US08/50378

§ 371 Date: Jul. 6, 2009

Related U.S. Application Data
(60) Provisional application No. 60/884,068, filed on Jan. 9, 2007.

(51) Int. Cl. C12N 1/19; A62D 3/02

(52) U.S. Cl. 435/254.21; 435/262.5

(57) ABSTRACT

The present disclosure relates to yeast biocatalysts and methods of using yeast biocatalysts for detoxifying a biowarfare agent. In some embodiments, a yeast biocatalyst may include a nucleic acid encoding a phage lysin operably linked to a prepro leader sequence, an expression control sequence operably linked to the nucleic acid, and a bioeffective amount (e.g., enough to detoxify) of the phage lysin. A yeast biocatalyst may include an engineered strain of a Saccharomyces cerevisiae. A phage lysin may be selected from the group consisting of PlyG and PlyPH. A method of detoxifying a biowarfare agent may include contacting a biowarfare agent with a yeast biocatalyst under conditions that permit detoxification. Conditions that permit detoxification, according to some embodiments, may include conditions that permit expression and secretion of a phage lysin. In some embodiments, conditions that permit detoxification may include conditions that permit germination of a spore. For example, a method of detoxifying a biowarfare agent spore may include contacting the spore with growth media (e.g., complete media or complex media) having alanine.

### RELATED APPLICATION

This application claims the benefit of provisional patent application entitled “Yeast Biocatalysts for Degradation of Biowarfare Agents,” Application Ser. No. 60/884,068 filed Jan. 9, 2007, the contents of which are incorporated herein in their entirety by reference.

### TECHNICAL FIELD

The present disclosure, according to some embodiments, relates to yeast biocatalysts that may ameliorate and/or eliminate a biohazard associated with a biowarfare agent.

### BACKGROUND

The threat of a biological tenor event against the US is credible. Since September 2001 beginning with the attacks on the world trade center, the subsequent deliberate dissemination of anthrax through the US postal system, and the continuing war on terror and military action against Iraq, has brought to the forefront the possibility of biological terrorism within the continental US. In addition, the likelihood that the target of choice is either a civilian ‘soft’ target or a symbolic building or organization is a distinct possibility. The most likely biological terrorist weapons include but are not limited to: (i) bacteria such as Bacillus anthracis, Yersinia pestis, Francisella tularensis, Brucella species, Burkholderia malleio, Salmonella species, Shigella species, Vibrio cholerae and Escherichia coli O157:H7; (ii) viruses such as the filoviruses (e.g., Ebola, Marburg), arenaviruses (e.g., Lassa, Machupo), alpha viruses (e.g., Venezuelan equine encephalitis, eastern equine encephalitis, western equine encephalitis) and (iii) toxins such as botulinum, Staphylococcal enterotoxins, ricin, and Clostridium perfringes episilon toxin.

### SUMMARY

Therefore, new agents that detoxify a biological terror threat (e.g., in liquids or solid surfaces (buildings)) are needed.

According to some embodiments, the disclosure relates to yeast biocatalysts for detoxifying a biowarfare agent. Detoxification may include any reduction in a toxic property of a biowarfare agent. In some embodiments, this may include degrading, sanitizing, killing, and/or eliminating the biowarfare agent. A yeast biocatalyst for detoxifying a biowarfare agent may include a nucleic acid encoding a phage lysin operably linked to a prepro leader sequence, an expression control sequence operably linked to the nucleic acid, and a bioeffective amount (e.g., enough to detoxify) of the phage lysin. A yeast biocatalyst may include an engineered strain of a Saccharomyces cerevisiae. In some embodiments, a yeast biocatalyst may comprise any lysin and/or any other bacteriocidal protein including, for example, staphylococcal AtlA, staphylococcal AtlE (60), Bacillus PlyB (58), streptococcal PlyC (59), Bacillus PlyG, Bacillus PlyPH (48), yersinia pesticin (61).

In some embodiments, the disclosure relates to methods of detoxifying a biowarfare agent. For example, a method of detoxifying a biowarfare agent may include contacting a biowarfare agent with a yeast biocatalyst under conditions that permit detoxification. Conditions that permit detoxification, according to some embodiments, may include conditions that permit expression and secretion of a phage lysin. In some embodiments, conditions that permit detoxification may include conditions that permit germination of a spore. For example, a method of detoxifying a biowarfare agent spore may include contacting the spore with growth media (e.g., complete media or complex media) having alanine.

The disclosure provides, in some embodiments, a composition for detoxifying a biowarfare agent comprising a yeast biocatalyst, a bacterial growth media; and alanine. A composition, according to some embodiments, for detoxifying a biowarfare agent may comprise a yeast biocatalyst growth media, e.g., spent media. Such media may include a buffer and/or a proteinase inhibitor.

### DETAILED DESCRIPTION

The present disclosure, according to some embodiments, relates to a biological decontamination system and method using one or more phage lysinsi.

The Center for Disease Control (CDC) has listed Bacillus anthracis (anthrax), Yersinia pestis (plague) and Francisella tularensis (tularemia) as Category A bacterial pathogens most likely to be used in a bioterrorist attack. Category A bacterial pathogens are defined as high-priority and high-risk agents since they: (i) can be easily disseminated or transmitted from person to person; (ii) result in high mortality rates and have the potential for major public health impact; (iii) might cause public panic and social disruption and; (iv) require special action for public health preparedness. Out of the three bacterial pathogens, B. anthracis is considered the most likely bioterrorist weapon of choice (1, 2).

B. anthracis is an aerobic, Gram-positive, spore-forming, nonmotile pathogen of animals and humans that can exist in two states; either as a vegetative cell, or as a spore. The vegetative cell, which is the replicative form, survives very poorly outside of the host. In contrast, spores which are formed under starvation conditions and are the infectious form, can survive in a dormant state for years and are extremely resistant to chemical and physical insult (3, 4). Spores germinate under conditions rich in amino acids and nutrients such as those encountered in blood and tissues (5). Upon germination, toxins are released (lethal toxin and edema toxin encoded on the pX02 virulence plasmid) resulting in hemorrhage, edema, necrosis, and septicemia, and ultimately organ failure and death (6, 7). ‘Naturally occurring’ anthrax, which is caused by inhalation, cutaneous, or gastrointestinal (GI) exposure to B. anthracis, is extremely rare in the U.S. For example, there have been only 18 cases of inhalation anthrax in the U.S. in the 20th century, of which most occurred in specialized risks groups such as goat hair mill or wool mill employees, or by accidental contamination of laboratory personnel (2). Similarly, only isolated cases of GI anthrax, caused by the ingestion of B. anthracis contaminated meat, have been reported in the U.S. (8, 9). Nevertheless, recent world events and terrorist threats have dramatically increased the risk of anthrax exposure, which is considered the most likely bioterrorist weapon of choice (1, 2). This was particularly evident in the autumn of 2001 when bioterrorist's released B. anthracis spores in the U.S. postal system which caused 18 confirmed cases of cutaneous and inhalation anthrax (10). The B. anthracis material had a high spore concentration, uniform particle size, low electrostatic charge, and was treated to reduce clumping. These characteristics indicated that the material was ‘weapon grade’ and most likely produced by skilled scientists. Although the total number of cases was relatively small, the mortality rate associated with inhalation anthrax was 45% and all patients exhibiting symptoms of toxemia died, despite receiving appropriate antimicrobial therapy (10). The potential impact of a major anthrax bioterrorist attack is substantial; an aerosolized release of 50 kg of spores upwind of a major city could kill or incapacitate 220,000 people (3). Compounding this threat, is the possibility of deliberately releasing engineered antibiotic (penicillin and tetracycline) resistant strains which were reportedly produced in the former Soviet Union (11).

B. anthracis decontamination. Existing processes for decontamination of B. anthracis spores are difficult. The spores are generally resistant to heat, desiccation, radiation, pressure and chemicals due to its thick proteinaceous coat, low water content and/or insoluble spore proteins (12, 13). Consequently, disinfectants such as alcohols, phenols, ionic and nonionic detergents, acids and alkalis have limited effectiveness (4).

Agents which have shown utility for the inactivation of spores include chlorine dioxide, ethylene oxide, formaldehyde, gluteraldehyde, hydrogen peroxide, and methylene bromide (14). These agents may be advantageous for large-scale decontamination; however, they have issues with material compatibility, ventilation requirements, and toxicity. For example, chlorine dioxide oxidizes the spore outer coat and thereby prevents spore germination and was used as a decontaminant following the anthrax attacks of 2001. However, chlorine dioxide is unstable, corrosive and requires specific conditions for use. The Hart Senate office building in Washington required 4 attempts at decontamination with chlorine dioxide gas until it finally worked at a cost of $14-20 million; the Brentwood postal facility decontamination process required a year of planning, 2,000 pounds of chemical, a relative humidity of 75%, a temperature of 75°, and cost $100 million(http://groups.msn.com/AAEA/anthrax.msnw). Therefore, strategies that are non-toxic and utilize a ‘natural’ method of decontamination are needed.

B. anthracis phage and lysins. According to some embodiments of the disclosure, use of phage-derived enzymes such as lysins may provide a natural and specific approach for the elimination of B. anthracis (15-17). B. anthracis phage are currently used as a standard clinical diagnostic tool by the CDC and various public health laboratories for the identification of B. anthracis (2). The lytic phage γ is particular suited to this process since it is specific for B. anthracis and has broad strain susceptibility (19). In a recent study, γ phage was able to infect and lyse 49 out of 51 B. anthracis strains collected from diverse geographical locations such as Pakistan, Canada, Argentina, England, U.S., and South Africa (20). Without being limited to any particular mechanism of action, y phage may lyse B. anthracis by the production of the PlyG lysin which hydrolyzes the peptidoglycan component of the cell wall resulting in the release of progeny phage. The PlyG enzyme was isolated and purified and demonstrated to function as a potent anti-anthrax bactericidal agent (21). As little as 2 U of the enzyme (equivalent to approximately 2 μg of protein) added to 1 ml of log phase cells caused a 17,000-fold decrease in viability within 20 s, and near sterilization within 2 min (10⁸-fold reduction in viability). The enzyme worked equally well with either non-capsulated or capsulated strains indicating that the capsule did not block access of PlyG to the cell wall. The enzyme could exert its lethal effect in different mediums (growth media, phosphate buffer, human blood) and was active against 10 B. anthracis strains from different clonal types isolated worldwide. PlyG lysin was also able to target germinating spores and rescue BALB/c mice from a potentially lethal Bacillus intraperitoneal model of infection. Importantly, in contrast to antibiotics such as novobiocin and streptomycin, spontaneous PlyG resistant mutants were not obtained even after repeated PlyG exposure (a frequency of <5×10−9). This indicated that the generation of PlyG resistant mutants was uncommon and probably attributed to the targeting of the lysin to the essential cell-wall component, peptidoglycan. The reason why lytic enzyme such as PlyG are so effective as antibacterial agents is unclear but may be due to their evolution and selection over millions of years to quickly lyse the cell wall in order to release progeny phage and hence multiply. Since lytic phage have been identified from virtually every known bacterial species, the corresponding lytic enzymes offer enormous potential for the treatment of pathogenic bacteria (16, 17). For example, the utility of lytic enzymes as antibacterial agents have also been demonstrated for Enterococcus faecalis, Enterococcus faecium, and Streptococcus pneumoniae (15, 22, 23).

A yeast biocatalyst of the disclosure may, according to some embodiments, biodegrade and/or detoxify a chemical agent (e.g., O-ethyl S-[2-diisopropylaminoethyl]methylphosphonothiolate) in addition to or instead of a biowarfare agent. Accordingly, the disclosures of International (PCT) Patent Application No. PCT/US2006/045761, filed Nov. 30, 2006 entitled “Differentially Fluorescent Yeast Biosensors for the Detection and Biodegradation of Chemical Agents” are incorporated herein, in their entirety, by reference.

In some embodiments, secretion and accumulation of a functional phage lytic enzyme (e.g., PlyG) into the surrounding environment may generate potent bactericidal conditions that may decontaminate B. anthracis by cell lysis. The yeast Saccharomyces cerevisiae may be chosen for this development since it offers many potential advantages in comparison to purified enzyme formulations and bacterial biocatalysts since yeast: (i) are robust and resistant to environmental extremes; (ii) are extremely cheap and easy to produce and maintain; (iii) are genetically well defined with a plethora of mutants available through the Saccharomyces Genome Deletion Project (Stanford); (iv) can effectively secrete functional heterologous proteins (24, 25); (v) are non-pathogenic, and (vi) are stable, readily lyophilized and exhibit good survival rates after ten years of storage (26, 27) enabling mass production and stockpiling. In addition, the yeast may proliferate when needed if delivered in appropriate growth medium and continually secrete the antibacterial agent to provide a limitless supply. An anti-anthrax yeast biocatalyst may be produced by generating a yeast strain which will secrete the phage lysin PlyG, as illustrated in Example 1. A gene encoding PlyG may be codon optimized for expression in yeast and fused to a prepro leader sequence to direct the lysin through the yeast secretory pathway. A S. cerevisiae strain which displays an oversecretion phenotype will be utilized to enhance the amount of enzyme in the external medium. Example 2 illustrates testing that may be performed to determine whether a yeast produces functional enzyme that can decontaminate B. anthracis. Thereafter, anthrax spores may be generated and the utility of the yeast biocatalyst to kill those spores may be analyzed in conjunction with spore germinating agents.

In some embodiments, a yeast biocatalyst may ameliorate and/or inactivate B. anthracis and one or more other biowarfare agents. For example, a yeast may include lytic enzymes for each bacterial target or prospective target desired. To target Gram-negative bacterial pathogens however, it may be desirable or necessary to deliver the lysins in conjunction with outer-membrane permeabilizers in order for the lysin to gain access to the inner peptidoglycan cell wall. For a yeast biocatalyst to decontaminate viruses or toxins, a yeast may be modified to secrete a common anti-viral enzyme or a common enzyme that targets multiple toxins.

A yeast biocatalyst may be safe to humans according to some embodiments. For example, yeast may be non-pathogenic, as illustrated by their routine use in the cooking and fermentation industries, and may pose little or no threat to the user.

In some embodiments, a yeast biocatalyst may be easily transported (e.g., on passenger aircraft) to the location of its intended use. Once at the site of its intended use, a biocatalyst may be amplified on site to produce as much biocatalyst as desired or required. A yeast catalyst may be stored in a dried state, for example, to minimize weight and/or volume. A dried yeast biocatalyst may include, in some embodiments, powdered growth medium. For example, a dried yeast biocatalyst may formulated to allow reconstitution by simply adding water.

A yeast biocatalyst, without being limited to any mechanism, may kill B. anthracis using a ‘natural’ strategy. Even so, it may be desirable or necessary to remove and/or kill the yeast after decontamination is complete. This may be achieved, according to some embodiments, by engineering the yeast to strictly require a specific nutrient or other chemical. A yeast biocatalyst of the disclosure may operate at a broader spectrum of environmental conditions (e.g., temperature) than a bacterial biocatalyst. A yeast biocatalyst (e.g., a lyophilized preparation), in some embodiments, may display survival rates of up to or more than 25% after a 10-year storage period.

### EXAMPLES

Some specific embodiments of the disclosure may be understood, by referring, at least in part, to the following examples. These examples are not intended to represent all aspects of the disclosure in its entirety. Variations will be apparent to one skilled in the art. The following Examples 1-3 are prophetic.

### Example 1

### Generation of a S. cerevisiae Strain Capable of Secreting the Anti-Anthrax Enzyme PlyG

A yeast mutant strain, which displays an over-secretion phenotype, may be genetically engineered to overexpress the γ phage plyg gene (encoding the lytic PlyG enzyme). The plyg gene may be codon optimized to ensure efficient expression in yeast and may be fused to a consensus leader peptide to direct the enzyme through the secretory pathway. SDS-PAGE may be performed to demonstrate that PlyG enzyme is present in enzyme lysates and as a secreted protein in the extracellular medium.

Whole cell yeast biocatalysts may offer an inexpensive, safe, self-contained biological system that may be used to decontaminate a biowarfare agent. S. cerevisiae may be particularly suited for these studies because it is genetically tractable, has been used extensively for the heterologous expression of proteins (29), and is generally more robust and ‘hardy’ than other microorganisms. S. cerevisiae exhibits survival rates of about 10-25% after a 10-year storage period following freeze-drying and preservation (26-28). Consequently, this may reduce the logistical burden of preparation, stockpiling, and use in diverse conditions. In contrast to enzymes formulations and chemical decontamination solutions, a live yeast is not a fixed quantity and may be amplified as required.

B. anthracis was chosen as a target for Example 1 since (a) it is a Category A bioterrorist pathogen, (b) it has been used recently as a bioterrorist weapon on American soil, and (c) is classed as the most likely biowarfare weapon of choice (1, 2). The PlyG lytic enzyme derived from the broad-host-range y phage may be used as the killing component which may be secreted by the yeast in order to decontaminate B. anthracis. The gene encoding PlyG may be fused to a synthetic prepro leader sequence to target the protein through the Golgi apparatus and secretion pathway. Heterologous protein secretion using synthetic sequences has resulted in up to 140 μg/ml of functional secreted protein (25, 30). A S. cerevisiae over-secreting mutant strain, which exhibits enhanced release of heterologous proteins into the external medium, may be used, e.g., to maximize PlyG secretion (31). The ability of S. cerevisiae to secrete functional heterologous proteins into the external medium is well established (32-36).

Construction of a signal secretion prepro peptide-plyg fusion. A signal secretion prepro peptide plyg fusion may be constructed and codon optimized for expression in yeast. A yeast expression plasmid containing the plyg fusion gene under the transcriptional control of the yeast GAL10 promoter may be constructed and transformed into a super-secreting S. cerevisiae mutant strain. The production of PlyG by recombinant S. cerevisiae cells may be assayed in enzyme lysates and the external medium by SDS-PAGE analysis.

The yeast secretion system has the capacity to fold, proteolytically process, glycosylate and secrete proteins. For example, S. cerevisiae secretes the alpha-mating factor up to 100 μg/ml (35). Secretion is mediated by a prepro leader sequence which may target a protein to the golgi apparatus and the secretion pathway. The alpha factor prepro leader is the classical leader and has been used for the secretion of numerous proteins in yeast as well as other species (32-34). Designed synthetic leader sequences have also shown utility and increased secretion of heterologous proteins (37). The synthetic prepro leader sequence MKVLIVLLAIFAALPLALA-QPVINTTVGSAAEGSLDKR-EA may be used to direct PlyG through the secretory pathway (25); the first dash represents a putative signal cleavage site, and the second dash, a Kex2p cleavage site for correct processing of the mature protein. A spacer peptide may be present between the leader sequence and the gene in order to optimize Kex2p processing of the fusion protein (38). The leader sequence may be fused to the gene encoding the PlyG lysin (Genbank #AF536823). PlyG is a relatively small protein of 673 amino acids and a predicted mass of about 27000 (Mᵣ 27 KDa). Analysis of the leader sequence and the plyg gene using the graphical codon usage analyzer (www.gcua.de) indicates that the fusion sequence contains a number of rarely used S. cerevisiae codons. Therefore, the prepro leader-plyg sequence may be codon optimized to ensure efficient expression in yeast. Codon and mRNA structural optimization and generation may be performed by Bio S&T Inc. The prepro leader-plyg fusion may be designed to incorporate the restriction endonuclease sites EcoRI and BglII, respectively at the 5′ and 3′ ends to ease cloning. In addition, a preferred yeast translation initiation sequence (5′-AAAAGTATG) may be placed immediately preceding the ATG (bold) start codon of the leader peptide (39).

Construction of a yeast PlyG expression plasmid. The prepro leader-plyg fusion gene may be directionally cloned into the EcoRI and BglII sites of the E. coli) S. cerevisiae expression vector pESC-URA (Stratagene) using standard molecular biology techniques (40) to create pPLYG-URA. The pESC-URA plasmid contains the yeast 2μ origin and the URA3 auxotrophic marker gene for growth and selection in S. cerevisiae, the ColE1 origin and β-lactamase gene for growth and selection in E. coli, the GAL10 regulatable promoter for repressed (dextrose) or induced (galactose) expression in S. cerevisiae, and a transcription termination sequence downstream of the GAL10 promoter. Cloning into the EcoRI and BglII sites will place plyg under the transcriptional control of the inducible GAL10 promoter which will permit the correlation of induced expression and secreted protein with B. anthracis killing (Example 2). Moreover, the optimization of the translation initiation signals and expression from the very strong GAL10 promoter (41), should ensure that the amount of PlyG lysin produced will not be a limiting factor. Cloning and propagation may be performed in E. coli XL1-Blue MRF′ (Stratagene). Diagnostic restriction endonuclease digestions and agarose gel electrophoresis may be used to verify the correct clone has been selected. The sequence of the fusion gene may be verified by deoxy dye terminator sequencing.

S. cerevisiae transformaton. A plyg gene may be stably integrated into the S. cerevisiae chromosome to generate a stable yeast biocatalyst; however, episomal expression of plyg may be sufficient to have anti-biowarfare agent activity. The S. cerevisiae mutant strain (MATa his3D1 leu2DO lys2DO ura3DO DMNN10) (ATCC4013604) may be used for these studies. The mutant strain has been deleted for MNN10, a gene encoding a mannosyltransferase, which is responsible for cell wall mannoprotein biosynthesis. The MNN10 mutant displays an increased ability to secrete heterologous proteins presumably due to the altered mannoprotein cell wall content which may impact the release of secretory proteins into the media (31). The S. cerevisiae mutant strain may be transformed with pPLYG-URA and the empty control vector pESC-URA using a lithium acetate (LiAc) transformation procedure (42). Briefly, an overnight stationary phase culture may be inoculated into 300 ml YPD (2% bacto-peptone, 1% yeast extract, 2% dextrose [glucose], pH 5.8) supplemented with 200 μg/ml geneticin and grown at 30° C. until an OD600 of 0.5 is reached. The culture may be washed in 50 ml TE buffer and resuspended in 1.5 ml of freshly prepared 1×TE/LiAc solution (10 mM Tris-HCl, 1 mM EDTA, 0.1 M LiAc, pH 7.5). Yeast competent cells (0.1 ml) may be added to plasmid (0.1 μg) and herring testes carrier DNA (0.1 mg), and an additional 0.6 ml of a TE/LiAc plus 40% PEG solution may be added. After 30 min incubation at 30° C., 70 μl of DMSO may be added and the mixture may be heat shocked at 42° C. for 15 min. The mixture may be pelleted and resuspended in 0.5 ml of TE. The mixture may be plated (100 μl) onto synthetic dropout (SD) dextrose agar medium lacking uracil (Clontech) and incubated at 30° C. until colonies appear (usually 2-4 days). A transformation efficiency of 10⁵-10⁶/μg DNA is typically obtained.

The ability of recombinant yeast to produce PlyG enzyme may be assessed with enzyme lysates and culture supernatants by SDS-PAGE. Enzyme lysates may be analyzed initially to verify that PlyG is being expressed. Culture supernatants will then be analyzed to demonstrate that the protein is present in culture supernatants and is being secreted by the recombinant yeast. The presence of functional, active PlyG may be demonstrated, for example, as described in Example 2.

Enzyme lysates may be prepared from yeast cells harboring the plasmid pPLYG-URA (PlyG+) or empty control plasmid pESC-URA (PlyG−). S. cerevisiae PlyG+ and PlyG− (control) cells will be grown in SD media containing 2% galactose (SDgal, pGAL10 inducing conditions) at 37° C. until an OD600 of approximately 0.5 is reached. The cultures may be harvested by centrifugation and enzyme lysates may be prepared by incubating the cells with Y-MER dialyzable lysis buffer (Pierce Biotechnology) for 20 min at room temperature (RT), followed by centrifugation at 24,000×g for 15 min. Total protein may be measured using Bradford's reagents and equal amounts of yeast lysates may be resolved by SDS-PAGE and analyzed for the presence of PlyG. As a positive control, purified PlyG may be analyzed concurrently. PlyG may be purified from E. coli cells harboring a PlyG expression vector as described previously (21). Briefly, washed cells may be lysed with chloroform to yield crude PlyG. PlyG may be passed through a HiTrap Q Sepharose XL column (Amersham), bound to a Mono S HR 5/5 column, and eluted in a linear gradient containing 1 M NaCl. SDS-PAGE is expected to indicate the presence of PlyG prepared from yeast cells harboring the pPLYG-URA plasmid at a size slightly higher than the bacterial-purified PlyG (Mᵣ 27 KDa) due to the presence of the unprocessed prepro leader peptide.

Culture supernatants may be analyzed for the presence of secreted PlyG. S. cerevisiae PlyG+ and PlyG− yeast cells may be grown in minimal media as described above for 24-36 h or an OD600 of approximately 2.0 is reached. Cell-free culture supernatants may be prepared by centrifugation and by passing the cultures through a 0.25 μm filter. Extracellular (secreted) proteins will then be concentrated from the culture supernatants by, for example, ultrafiltration. Proteins may be separated by SDS-PAGE and visualized by silver staining. Since yeast does not secrete many proteins and a minimal yeast nitrogen based media (lacking many proteins compared to complex media) may be used to grow the recombinant yeast, the presence of PlyG is expected to be visible. Using a similar strategy, Parekh et al. (25, 30) detected up to 140 μg/ml of secreted active bovine pancreatic trypsin inhibitor in culture supernatants. The PlyG protein may be purified from the concentrated yeast supernatants, for example, for functional studies described in Example 2 using a strategy similar for the purification of the PlyG from bacterial cultures.

It may be determined that the amount of PlyG secreted by a recombinant yeast is or may be sub-optimal. To maximize the amount of secreted protein, a specific yeast mutant which has demonstrated an enhanced ability to secrete proteins may be used (31). Other mutants that display a super secreting phenotype (36) may be used. The Saccharomyces genome genome project has successfully deleted 95% of the yeast's 6,200 open reading frames and provided a wealth of information and mutants. Therefore, in other embodiments, other yeast mutants and/or alternative prepro leader secretion peptides may be utilized to maximize PlyG (or other lysin) secretion (24).

Episomal expression of the PlyG protein from a yeast 2μ plasmid may be used in this example.

In other embodiments, a plyg gene (or other lysin gene) may be stably integrated into a yeast genome. Such stable integration may result in a stable, clonal yeast population that maintains the inserted DNA sequence for many generations even in the absence of selective pressure (30, 44). Since the number of integrated copies may be proportional to the number of target sites in the yeast genome (51), yeast cells carrying multiple copies of the integrated DNA may be generated when the insertion sequence is present in multiple copies. Therefore, a gene encoding PlyG may be targeted by homologous recombination to the ribosomal DNA (rDNA) locus which encompasses about 140 copies of a 9.1 kb unit repeated in tandem on chromosome XII (52). By targeting the rDNA locus, phosphoglycerate kinase (PGK) was integrated at 100-200 copies per cell, and when expressed from the glyceraldehyde-3-phosphate dehydrogenase (GAPDH) promoter, represented approximately 50% of the total soluble protein (44). The GAPDH promoter may be used to drive PlyG expression since it is both strongly and constitutively expressed (30, 44, 53, 54). The use of optimal expression signals, the integration of multiple copies of the gene encoding PlyG into the yeast genome, and the generation of a stable yeast strain which maintains the plyg gene even in the absence of selective pressure, will generate an efficient yeast biocatalyst that can function outside the laboratory.

### Example 2

### Confirming that the Yeast Biocatalyst Secretes Functional PlyG which Efficiently and Quickly Kills B. anthracis

Purified PlyG and S. cerevisiae culture supernatants may be incubated with vegetatively growing B. anthracis. The attenuated B. anthracis Sterne 34F2 strain may be used to omit the safety and regulatory issues of using a virulent select agent. The functionality of culture supernatants containing the secreted PlyG enzyme to decontaminate B. anthracis may be analyzed by phase contrast microscopy, spectrophotometric measurements at an optical density of 600 nm (OD600), and colony forming ability (CFUs). The kinetics of killing may be assessed by measuring the time from initial contact to loss of bacterial viability. The incubation time required to generate sufficient functional amounts of PlyG in the culture medium may be measured.

The PlyG lytic enzyme derived from the broad-host-range B. anthracis γ phage may be used as the killing component to decontaminate B. anthracis. Phage lytic enzymes such as PlyG have evolved over millions of years to quickly and efficiently lyse bacterial cell walls in order to release the newly formed progeny phage. The enzyme targets the cell wall by hydrolyzing the peptidoglycan component, which is generic to all bacteria. As little as 2 μg of PlyG can cause a 10⁸-fold reduction in cell viability after a 2 min incubation (21). Importantly, PlyG functions against isolates collected from diverse worldwide locations and thus displays broad-strain activity. The broad-strain activity of PlyG is not unexpected since it is derived from γ phage which lyses nearly all B. anthracis isolates including both rough and smooth (encapsulated) strains.

Recombinant yeast cells harboring pPLYG-URA may secrete functional PlyG which can efficiently and quickly kill B. anthracis.

The ability of secreted PlyG to kill B. anthracis may be analyzed. Purified PlyG derived from Example 1 and cell-free culture supernatants from PlyG+ S. cerevisiae may be incubated with vegetatively growing B. anthracis. The attenuated B. anthracis Sterne 34F2 strain may be used to omit the safety and regulatory issues of using a virulent select agent. The functionality of supernatants containing the secreted PlyG enzyme to kill B. anthracis may be analyzed by phase contrast microscopy, spectrophotometric measurements (OD600), and colony forming ability (CFUs). The kinetics of killing may be assessed by measuring the time from initial contact to loss of bacterial viability. The incubation time required to generate sufficient amounts of functional PlyG in the culture medium to mediate killing may be measured.

B. anthracis killing assays. The ability of yeast PlyG+ culture supernatants, and purified PlyG (prepared from yeast culture supernatants) to kill vegetative B. anthracis may be assessed in order to: (i) demonstrate that the secreted PlyG is functionally active, and/or (ii) demonstrate that the recombinant yeast secretes functional PlyG in sufficient quantities to kill B. anthracis. The attenuated B. anthracis Sterne 34F2 strain (Colorado Serum company Cat # 19102) may be used for these experiments.

Virulent B. anthracis strains possess 2 virulence plasmids which are required for infection; the pX01 which controls the synthesis of the tripartite exotoxins (protective antigen, edema factor, and a lethal factor) and pX02, which is responsible for the synthesis of the poly-D-glutamic acid capsule. B. anthracis Sterne 34F2 strain lacks the pX02 encapsulating plasmid, and is commonly used as a less virulent surrogate for in vitro studies (45-47).

Cell-free culture supernatants may be prepared from recombinant PlyG+ and PlyG− yeast. Cell free supernatants, as opposed to yeast cultures, may be used in the B. anthracis killing assays in order to measure changes in OD600 and CFU attributed solely to B. anthracis. S. cerevisiae PlyG+ and PlyG− cells may be grown in SD media containing 2% galactose (SDgal, pGAL10 inducing conditions) at 37° C. for approximately 24 h or until the culture is saturated. The culture medium may be buffered with 100 mM HEPES pH 7.5 in order to maintain the culture at near neutral pH. Yeast cells may be collected by centrifugation at 2, 500×g for 10 min and the resulting supernatant may be passed through a 0.25 μm filter to generate PlyG+ and PlyG− cellfree supernatants. B. anthracis Sterne may be grown in Brain Heart Infusion (BHI) medium until an OD600 of 0.6.

The exponentially growing vegetative cells may be divided into 4 equal aliquots and harvested by centrifugation at 4,000×g for 10 min. The resulting pellets may be resuspended with:
(i) PlyG− cell-free supernatants (negative control);
(ii) PlyG− cell-free supernatants containing 2 μg/ml of the purified PlyG prepared from bacterial cultures (Example 1, positive control). This positive control will demonstrate the ability of the purified PlyG to function in ‘spent’ yeast culture supernatants. The equivalent of 2 μg/ml of bacterial purified PlyG caused a 17,000-fold decrease in B. anthracis viability within 20 s and a 10⁸ decrease in 2 min, (17, 21);
(iii) PlyG− cell-free supernatants containing 2 μg/ml of the purified secreted PlyG prepared from yeast culture supernatants (Example 1). This will demonstrate the activity of the yeast PlyG; or
(iv) PlyG+ cell-free supernatants, which will demonstrate the ability of the yeast enzyme to function in ‘spent’ medium.

The OD600 and cell viability of the cultures may be measured at time zero and in 5 min intervals for 30 min to determine how quickly the yeast purified PlyG, and culture supernatants containing secreted PlyG, can lyse B. anthracis. Cell viability may be measured by plating out serially diluted B. anthracis cells (and hence PlyG enzyme) on BHI agar plates, and counting colony forming units (CFUs) after 24-48 h growth at 37° C. Phase contrast microscopy may also be utilized to look for characteristic changes in cell morphology. Changes in the phenotypic appearance from filamentous cells to rod-like forms, and the formation of “ghost” cells due to the loss of cytoplasmic material (cell lysis) are anticipated (21). The results are expected to demonstrate that: (i) PlyG purified from yeast culture supernatants is functional and can lyse B. anthracis; (ii) unprocessed yeast culture supernatants contain secreted PlyG in sufficient quantities to lyse B. anthracis, and (iii) PlyG+ supernatants can quickly lyse B. anthracis.

The speed with which PlyG is able to lyse B. anthracis is an important component of the efficiency of the biocatalyst. The time required to generate sufficient quantities of secreted PlyG in the surrounding medium to mediate killing is also an essential consideration for the efficiency of the biocatalyst. Therefore, S. cerevisiae PlyG+ and PlyG− cells may be grown in buffered SDgal at 37° C. until an OD600 of 0.8. Yeast cells may be collected by centrifugation (2,500×g for 10 min), washed with an equal volume of phosphate buffered saline, and resuspended in fresh medium. Aliquots of the culture supernatant may be assayed for functional PlyG at 30 min intervals. Cell-free culture supernatants may be collected and incubated with exponentially growing vegetative B. anthracis as described above. The time required to generate functional PlyG+ supernatants may be assessed by changes in B. anthracis OD600 and cell viability compared to supernatants lacking PlyG. It is expected that B. anthracis viability will decrease as the time increases due to the accumulation of PlyG in the medium.

The use of S. cerevisiae to secrete functional heterologous proteins into the external medium is well established (32-36). Various mammalian proteins have been secreted from yeast at levels between 50 and 200 μg/ml (35). Since PlyG is not a large protein (Mᵣ 27 KDa) and is an extremely efficient lysin, the secretion efficiency is expected to generate sufficient PlyG to mediate B. anthracis killing. The PlyG lysin is functional in bacterial growth medium, is partially active in blood and is able rescue mice from a lethal Bacillus infection (17, 21). This suggests that PlyG is fairly stable.

Yeast media (e.g., spent media) may include one or more extracellular proteases or other materials (e.g., waste products) that interfere with lysin function. For example, the pH of spent media may be from about 4 to about 5. Therefore, in some embodiments, a media may be treated to improve lysin function. This may include removing a protease, adding one or more protease inhibitors, and/or supplementing SDgal with 100 mM HEPES buffer pH7.5. Alternatively, a different B. anthracis lysin which displays activity over a range of pH values and temperatures may be utilized. For example, PlyPH is a B. anthracis lytic enzyme which displays activity at pH's as low as 4 and as high as 10. PlyPH also retains full activity at 4° C. and 60° C. and in relatively high salt concentrations (150 mM) (48).

### Example 3

### Assessing Effectiveness of a Yeast Biocatalyst to Decontaminate B. Anthracis Spores

The ability of the yeast biocatalyst to function under conditions conducive for spore germination may be analyzed.

Spores are the infectious and weaponized form that may be used in a biowarfare/bioterrorist attack (1-3). B. anthracis spores are formed under starvation conditions and are extremely resistant to physical and chemical insult (4). The spores germinate under conditions rich in amino acids and nutrients which are encountered in the human host. Upon germination, toxins may be released (lethal toxin and edema toxin) that cause hemorrhage, edema, and necrosis. Inhalation symptoms include fever and chills, malaise, cough, nausea, and chest discomfort; 99% of untreated infected people die.

Decontamination of bacterial spores may be a difficult process. Most disinfectants such as detergents, alcohols and phenols are ineffective against spores. PlyG lyses vegetative cells and germinating spores; however, spores may be resistant to PlyG-induced lysis since the peptidoglycan cell wall is protected by a thick proteinaceous outer spore coat (17, 21). In order to target spores and overcome this limitation, a yeast biocatalyst may be delivered in combination with amino acids or nutrients that elicit spore germination. Spores may be induced to germinate quickly by the presence of amino acids such as L-alanine (49, 50). Therefore, the efficiency and feasibility of the yeast biocatalyst to decontaminate B. anthracis spores may be analyzed when delivered in conjunction with spore germination agents.

B. anthracis Sterne spores may be generated. The ability of spores to germinate in fresh medium, and in culture supernatants (spent medium) in the presence of L-alanine may be analyzed. The ability of the yeast PlyG+ biocatalyst to kill B. anthracis spores when used in conjunction with spore germinating agents may be determined.

B. anthracis spore generation. A single B. anthracis Sterne colony may be grown overnight in BHI medium supplemented with 0.5% glycerol at 37° C. with vigorous shaking. Spores may be formed under conditions of nutrient starvation. Therefore, spores may be generated by diluting the overnight culture 1:10 in minimal media (0.5 mM MgCl₂.6H₂O, 0.01 mM MnCl₂.4H₂O, 0.05 mM FeCl₃.6H₂O, 0.05 mM ZnCl₂, 0.2 mM CaCl₂.6H₂O, 13 mM KH₂PO₄, 26 mM K₂HPO₄, 20 μg/ml glutamine, 1 mg/ml acid casein hydrolysate, 1 mg/ml enzymatic casein hydrolysate, 0.4 mg/ml enzymatic yeast extract, and 0.6 mg/ml glycerol) and incubating at 37° C. with shaking (49, 50). After 48 h, phase contrast microscopy may be used to indicate the presence of refractile spores. Spores are small (1 μm) and are ‘phase bright’ compared to the larger vegetative cells (1-8 μm long, by 1-1.5 μm wide); cultures are expected to consist of >90% spores. Cultures may be centrifuged for 30 min at 1,500×g, washed 3 times with sterile dH₂O, and resuspended in 1 ml of dH₂O. Following a 30 min incubation at 65° C. (which kills the vegetative form only), the spores may be washed 4 times with dH₂O, with the uppermost layer of the pellet discarded after each wash. Using phase contrast microscopy, the resulting suspension is expected to consist of >95% spores with minimal vegetative debris. Spores may be enumerated using a haemocytometer and by colony counting after growth on BHI agar at 37° C. The spores may be stored at room temperature until needed.

Spore germination. Spores may be formed under starvation conditions, but germinate under conditions rich in nutrients and amino acids. In particular, B. anthracis spores are readily, and quickly germinated in media supplemented with the amino acid L-alanine (47, 49, 50). In order to determine the efficiency of spore germination under conditions compatible with the yeast biocatalyst, 1×10⁷ B. anthracis spores may be resuspended at RT in either: (i) fresh SDgal buffered with 50 mM HEPES (pH 7.5); (ii) fresh buffered SDgal supplemented with 100 mM L-alanine; (iii) cell-free buffered SDgal media (‘spent’) prepared from saturated wild-type yeast cultures, and (iv) cell-free buffered SDgal media (‘spent’) prepared from saturated wild-type yeast cultures supplemented with 100 mM L-alanine. The efficiency of germination may be monitored by phase contrast microscopy, by changes in absorbance at OD450 nm, and by CFUs after heat treatment at 65° C. for 30 min (only germinating spores may be heat sensitive, while non-germinating spores may be resistant). Spore germination is expected to be evident under all conditions analyzed but greatest in media supplemented with L-alanine. If required, the spent medium may be supplemented with other agents such as serum to enhance germination (45). Under optimal conditions, >95% germination may be obtained within 15 min (49).

Spore killing assays. The ability of the yeast biocatalyst to kill B. anthracis spores may be determined under the spore germination conditions determined above. 1×10⁷ B. anthracis spores may be resuspended in either buffered cell-free PlyG+, or PlyG− supernatants, supplemented with L-alanine as required. The ability of yeast PlyG+supernatants to lyse germinating spores may be compared to control supernatants. The time from initial contact to loss of cell viability may be assessed by plating serially diluted B. anthracis and measuring CFUs after 24-48 h growth on BHI agar medium at 37° C.

Decontamination may be enhanced by efficient spore germination. Minimal (SDgal) media supplemented with germinating agents may be used to induce spore germination. Since spore germination may be triggered by the availability of nutrients, complex media may be used, for example, with biocatalysts having a chromosomally integrated lysin gene. Growth and selection may be mediated by an antibiotic resistant marker (e.g., geneticin) in complex media such as YPD which may be more conducive for spore germination.

### REFERENCES

Each of the following documents is incorporated herein, in its entirety, by reference.
1. Greenfield, R. A. & Bronze, M. S. (2003) Drug Discov Today 8, 881-8.
2. Inglesby, T. V., O'Toole, T., Henderson, D. A., Bartlett, J. G., Ascher, M. S., Eitzen, E., Friedlander, A. M., Gerberding, J., Hauer, J., Hughes, J., McDade, J., Osterholm, M. T., Parker, G., Peri, T. M., Russell, P. K. & Tonat, K. (2002) Jama 287, 2236-52.
3. Wenzel, R. P. (2002) Trans Am Clin Climatoli Assoc 113, 42-53; discussion 53-5.
4. Erickson, M. C. & Kornacki, J. L. (2003) J Food Prot 66, 691-9.
5. Lyons, C. R., Lovchik, J., Hutt, J., Lipscomb, M. F., Wang, E., Heninger, S., Berliba, L. & Garrison, K. (2004) Infect Immun 72, 4801-9.
6. Ascenzi, P., Visca, P., Ippolito, G., Spallarossa, A., Bolognesi, M. & Montecucco, C. (2002) FEBS Lett 531, 384-8.
7. Oncu, S., Oncu, S. & Sakarya, S. (2003) Med Sci Monit 9, RA276-83.
8. (2000) Jama 284, 1644-6.
9. Beatty, M. E., Ashford, D. A., Griffin, P. M., Tauxe, R. V. & Sobel, J. (2003) Arch Intern Med 163, 2527-31.
10. Bartlett, J. G., Inglesby, T. V., Jr. & Borio, L. (2002) Clin Infect Dis 35, 851-8.
11. Alibek, K. & Handelman, S. (1999) Biohazard: The Chilling True Story of the Largest Covert Biological Weapons Program in the World. (Random House.
12. Nicholson, W. L., Munakata, N., Horneck, G., Melosh, H. J. & Setlow, P. (2000) Microbiol Mol Biol Rev 64, 548-72.
13. Setlow, B. & Setlow, P. (1993) Appl Environ Microbiol 59, 3418-23.
14. Spotts Whitney, E. A., Beatty, M. E., Taylor, T. H., Jr., Weyant, R., Sobel, J., Arduino, M. J. & Ashford, D. A. (2003) Emerg Infect Dis 9, 623-7.
15. Fischetti, V. A. (2003) Ann N Y Acad Sci 987, 207-14.
16. Fischetti, V. A. (2001) Nat Biotechnol 19, 734-5.
17. Fischetti, V. A. (2005) in Bacteriophages: biology and applications, eds. Kutter, E. & Sulakvelidze, A. (CRC Press, Boca Raton), pp. 321-334.
18. Cowles, P. B. (1931) J Bacteriol 21, 161-6.
19. Brown, E. R. & Chemy, W. B. (1955) J Infect Dis 96, 34-9.
20. Abshire, T. G., Brown, J. E. & Ezzell, J. W. (2005) J Clin Microbiol 43, 4780-8.
21. Schuch, R., Nelson, D. & Fischetti, V. A. (2002) Nature 418, 884-9.
22. Yoong, P., Schuch, R., Nelson, D. & Fischetti, V. A. (2004) J Bacteriol 186, 4808-12.
23. Loeffler, J. M., Nelson, D. & Fischetti, V. A. (2001) Science 294, 2170-2.
24. Eiden-Plach, A., Zagorc, T., Heintel, T., Carius, Y., Breinig, F. & Schmitt, M. J. (2004) Appl Environ Microbiol 70, 961-6.
25. Parekh, R., Forrester, K. & Wittrup, D. (1995) Protein Expr Purif 6, 537-45.
26. Miyamoto-Shinohara, Y., Imaizumi, T., Sukenobe, J., Murakami, Y., Kawamura, S. & Komatsu, Y. (2000) Cryobiology 41, 251-5.
27. Lodato, P., Se govia de Huergo, M. & Buera, M. P. (1999) Appl Microbiol Biotechnol 52, 215-20.
28. Diniz-Mendes, L., Bernardes, E., de Araujo, P. S., Panek, A. D. & Paschoalin, V. M. (1999) Biotechnol Bioeng 65, 572-8.
29. Wiseman, A. (1996) Endeavour 20, 130-2.
30. Parekh, R. N., Shaw, M. R. & Wittrup, K. D. (1996) Biotechnol Prog 12, 16-21.
31. Bartkeviciute, D. & Sasnauskas, K. (2004) FEMS Yeast Res 4, 833-40.
32. Botstein, D. & Fink, G. R. (1988) Science 240, 1439-43.
33. Romanos, M. A., Scorer, C. A. & Clare, J. J. (1992) Yeast 8, 423-88.
34. Bitter, G. A., Chen, K. K., Banks, A. R. & Lai, P. H. (1984) Proc Natl Acad Sci U S A 81, 5330-4.
35. Moir, D. T. (1989) Biotechnology 13, 215-31.
36. Sakai, A., Shimizu, Y. & Hishinuma, F. (1988) Genetics 119, 499-506.
37. Kjeldsen, T., Hach, M., Balschmidt, P., Havelund, S., Pettersson, A. F. & Markussen, J. (1998) Protein Expr Purif 14, 309-16.
38. Kjeldsen, T., Brandt, J., Andersen, A. S., Egel-Mitani, M., Hach, M., Pettersson, A. F. & Vad, K. (1996) Gene 170, 107-12.
39. Looman, A. C. & Kuivenhoven, J. A. (1993) Nucleic Acids Res 21, 4268-71.
40. Sambrook, J., Fritsch, E. F. & Maniatis, T. (1989) Molecular cloning: a laboratory manual. (Cold Spring Harbor Press, New York).
41. Peleg, Y., Rokem, J. S., Goldberg, I. & Pines, O. (1990) Appl Environ Microbiol 56, 2777-83.
42. Ito, H., Fukuda, Y., Murata, K. & Kimura, A. (1983) J Bacteriol 153, 163-8.
43. Murray, A. W. & Szostak, J. W. (1983) Cell 34, 961-70.
44. Lopes, T. S., Klootwijk, J., Veenstra, A. E., van der Aar, P. C., van Heerikhuizen, H., Raue, H. A. & Planta, R. J. (1989) Gene 79, 199-206.
45. Ireland, J. A. & Hanna, P. C. (2002) Infect Immun 70, 5870-2.
46. Schuch, R. & Fischetti, V. A. (2006) J Bacteriol 188, 3037-51.
47. Titball, R. W. & Manchee, R. J. (1987) J Appl Bacteriol 62, 269-73.
48. Yoong, P., Schuch, R., Nelson, D. & Fischetti, V. A. (2006) J Bacteriol 188, 2711-4.
49. Ireland, J. A. & Hanna, P. C. (2002) J Bacteriol 184, 1296-303.
50. Stewart, G. S., Johnstone, K., Hagelberg, E. & Ellar, D. J. (1981) Biochem J 198, 101-6.
51. Wilson, J. H., Leung, W. Y., Bosco, G., Dieu, D. & Haber, J. E. (1994) Proc Natl Acad Sci USA 91, 177-81.
52. Petes, T. D. (1979) Proc Natl Acad Sci USA 76, 410-4.
53. Edens, L., Born, I., Ledeboer, A. M., Maat, J., Toonen, M. Y., Visser, C. & Verrips, C. T. (1984) Cell 37, 629-33.
54. Imamura, T., Araki, M., Miyanohara, A., Nakao, J., Yonemura, H., Ohtomo, N. & Matsubara, K. (1987) J Virol 61, 3543-9.
55. Novosel'tsev, N. N., Marchenkov, V. I., Kravchenko, A. N., Valentsev, V. E. & Tinker, L. A. (1990) Zh Mikrobiol Epidemiol Immunobiol, 15-8.
56. Brussow, H. & Kutter, E. (2005) in Bacteriophages: Biology and Applications, eds. Kutter, E. & Sulakvelidze, A. (CRC Press, Boca Raton).
57. Cheng, Q., Nelson, D., Zhu, S. & Fischetti, V. A. (2005) Antimicrob Agents Chemother 49, 111-7.
58. Porter C J, Schuch R et al., 2006, J. Mol. Biol. PMID: 17182056.
59. Nelson D, Schuch R, Chahales P, Zhu S, Fischetti V A, 2006, Proc. Natl. Acad. Sci. 103(28):10765-70.
60. Biswas R, Voggu L, Simon U K, Hentschel P, Thumm G, Gotz F, 2006, FEMS Microbiol Lett. 259(2):260-8.
61. Vollmer W, Pilsl H, Hantke K, Holtje J V, Braun V., 1997, J. Bacteriol. 179(5):1580-3

1. A yeast biocatalyst for detoxifying a biowarfare agent, said yeast biocatalyst comprising:
a nucleic acid encoding aphage lysin operably linked to a prepro leader sequence;
an expression control sequence operably linked to the nucleic acid; and
a bioeffective amount of thephage lysin,
wherein the yeast is Saccharomyces cerevisiae,
wherein thephage lysin is operable to detoxify a biowarfare agent,
wherein the lysin is selected from the group consisting of PlyG and PlyPH, and
wherein the biowarfare agent is a Bacillus anthracis spore.

2. A composition for detoxifying a biowarfare agent, said composition comprising:
a yeast biocatalyst having:
a nucleic acid encoding aphage lysin operably linked to a prepro leader sequence;
an expression control sequence operably linked to the nucleic acid; and
a bioeffective amount of thephage lysin, wherein the yeast is Saccharomyces cerevisiae, wherein thephage lysin is operable to detoxify a biowarfare agent, wherein the lysin is selected from the group consisting of PlyG and PlyPH, and wherein the biowarfare agent is a Bacillus anthracis spore.
a bacterial growth media; and
alanine.

3. A method of detoxifying a biowarfare agent, said method comprising:
contacting a biowarfare agent with a yeast biocatalyst under conditions that permit detoxification,
wherein the biowarfare agent is a Bacillus anthracis spore, wherein the yeast catalyst is Saccharomyces cerevisiae, having:
a nucleic acid encoding aphage lysin operably linked to a prepro leader sequence;
an expression control sequence operably linked to the nucleic acid; and
a bioeffective amount of thephage lysin, wherein thephage lysin is operable to detoxify a biowarfare agent, wherein the lysin is selected from the group consisting of PlyG and PlyPH.

4. A method of detoxifying a biowarfare agent according to claim 3 further comprising contacting the Bacillus anthracis spore with growth media having alanine, wherein the growth media having alanine permits spore germination.
